(19) United States
(12) Patent Application Publication
(10) Pub. No.: US 2018/0133366 A1
(43) Pub. Date: May 17, 2018

(54) FLOWABLE TISSUE COMPOSITIONS WITH ENHANCED BUOYANCY PROPERTIES CONTAINING ANTIBIOTICS AND DERIVATIVES THEREOF

(71) Applicant: Neil RIORDAN, Westlake, TX (US)

(72) Inventor: Neil RIORDAN, Westlake, TX (US)

(21) Appl. No.: 15/814,247

(22) Filed: Nov. 15, 2017

Related U.S. Application Data
(60) Provisional application No. 62/423,193, filed on Nov. 16, 2016.

(51) Int. Cl. A61L 27/36; A61L 27/50; A61L 27/38; A61K 35/39; A61K 35/407; A61K 35/35; A61K 35/50; A61K 35/51; A61K 9/00

(52) U.S. Cl. CPC A61L 27/3687; A61L 27/50; A61L 27/3691; A61L 27/3804; A61K 35/39; A61K 35/407; A61L 2300/232; A61K 35/50; A61K 35/51; A61K 9/0019; A61L 2400/06; A61L 2300/406; A61L 2300/214; A61K 35/35

(57) ABSTRACT

Disclosed are compositions of matter, protocols, and treatment means for generation of tissues capable of administration in a flowable manner through contain with one or more antibiotics, or derivatives of said antibiotics. In one embodiment the invention provides the treatment of morselized amniotic membranes with antibiotics in a manner as to increase buoyancy of morselized components of said amniotic membranes in order to prevent gravity based accumulation of said morselized tissue. In another embodiment buoyancy of morselized tissue is modulated by contacting said tissue with an antibiotic or derivative thereof in a manner to allow for increased uniformity of said morselized tissue components throughout a vessel. In another embodiment, pancreatic islets are isolated and stored in a manner to prevent gravity based aggregation. The invention pertains to all tissues, with emphasize in a preferred embodiment to tissues which are useful when administered in a flowable manner.

### CROSS-REFERENCE TO RELATED APPLICATIONS

The present invention claims priority to U.S. Provisional Application No. 62/423,193, filed Nov. 16, 2016, which is hereby incorporated in its entirety including all tables, figures, and claims.

### FIELD OF THE INVENTION

The invention pertains to the field of biological preparations, more particularly the invention relates to the field of tissue preparations modified to increase flowability in order to augment ability for injection into a recipient. More particularly the invention relates to the field of modifying digested tissue in a manner so as to modulate buoyancy of said tissue in order to provide a more uniform distribution of said tissue in a chamber so as to prevent aggregation of said tissue.

### BACKGROUND OF THE INVENTION

The clinical implementation of cellular therapy is considered one of the major advancements in medicine in the sense that in contrast to artificial interventions, cells possess a homeostatic ability to support natural processes associated with healing and achievement of equilibrium in physiological systems. Although cellular therapy was originally used in the 1900s, pioneered by Paul Neihans [1], who developed various means of administering cells to support deficiencies in the body, this approach fell out of favor due to uncharacterized nature of injectable products, as well as variation between preparations [2, 3]. The modern era of cell therapy began with the introduction of bone marrow transplants for treatment of hematopoietic disorders [4]. Subsequent advancements in cell therapy included the clinical introduction of “tissue repair” cells in the form of mesenchymal stem cells (MSC) [5], development of pancreatic islet transplantation as a means of treating Type 1 diabetes [6], dendritic cell therapies for cancer [7, 8], and autoimmunity [9, 10], as well as T cell therapies [11-13].

While the majority of cellular therapies currently in use call for administration of cellular suspensions, in some instances it is desirable to administer cells as aggregates. Aggregates of cells possess several advantages to administration of mononuclear cells including retaining a cellular environment allowing for cells to maintain viability for longer amounts of time subsequent to injection, protection from host mediated rejection factors, and permitting production of growth factors for an extended time period.

Examples of uses of cellular aggregates include administration of pancreatic islets, in which said islets are comprised not only of insulin producing beta cells but also alpha and gamma cells. Other examples of transplantation of cellular aggregates include amniotic membrane grafts. In one study, administration of particulates of amniotic membrane combined with umbilical cord particulates was useful in regenerating cartilage in a model of osteoarthritis

Clinical trials of injectable amniotic membrane particulates whether fresh or cryopreserved, have shown benefit in conditions including plantar fascititis [15, 16], corneal epithelial defects [17], chronic wounds [18], and various ocular conditions [19].

Unfortunately, during preparation of suspensions of cellular aggregates, in many occasions, cellular aggregates deposit to the bottom of the flask or vessel in which they are placed. The invention teaches means of overcoming this through altering the buoyancy of said cellular aggregates.

### DESCRIPTION OF THE INVENTION

The invention teaches means of treating aggregates of cells with agents to alter buoyancy of said aggregates in order to allow said aggregates to maintain an even distribution in a vessel. One particular embodiment of the invention teaches that treatment of cellular aggregates with antibiotics results in modulation of buoyancy. In one particular embodiment, the invention teaches that morselized pieces of amniotic membrane may be treated with a mixture of Gentamicin Sulfate, Penicillin, Streptomycin and Amphotericin B. The combination of antibiotics may be used together or as individual agents. In one particular embodiment, an antibiotic cocktail is generated comprising of: a) Gentamicin Sulfate 50 ug/ml; b) Penicillin 100 U/ml; c) Streptomycin 100 ug/ml and d) Amphotericin B 250 ng/ml. Said cocktail is considered 100%. Pieces of amniotic membrane are placed in the cocktail at a concentration of 50% antibiotic cocktail and 50% phosphate buffered saline (PBS). The amniotic membrane pieces are subsequently placed in an orbital shaker for 1 hour. After one hour, the tissue was rinsed three times in PBS. After the final rinse, the tissue was centrifuged for 5 minutes at 800g. Tissue is then milled to observe buoyancy in PBS. Neutral buoyancy is observed using the 50% stock of the cocktail. By endowing neutral buoyancy upon amniotic membrane tissue pieces the procedure of contacting with antibiotics allows for prevention of tissue aggregation and facilitates more effective ability to inject said tissue fragments.

As used herein, “human cells, tissues, or cellular or tissue-based products (HCT/Ps)” means articles containing or consisting of human cells or tissues that are intended for implantation, transplantation, infusion, or transfer into a human recipient.

As used herein, “human tissue” means any tissue derived from a human body. In some embodiments, the human tissue is amniotic membrane.

As used herein, “tissue graft” means a matrix of proteins (e.g., collagen and elastin) and glycans (e.g., dermatan, hyaluronan, and chondroitin) that is used to replace damaged, compromised, or missing tissue. In certain instances, the matrix is laid down and host cells gradually integrate into the matrix.

As used herein, “minimal manipulation” means (1) for structural tissue, processing that does not alter the original relevant characteristics of the tissue relating to the tissue's utility for reconstruction, repair, or replacement; and (2) for cells or nonstructural tissues, processing that does not alter the relevant biological characteristics of cells or tissues.

As used herein, “homologous use” means the repair, reconstruction, replacement, or supplementation of a recipient's cells or tissues with an HCT/P that performs the same basic function or functions in the recipient as in the donor.

As used herein, “processing” means any activity performed on an HCT/P, other than recovery, donor screening, donor testing, storage, labeling, packaging, or distribution, such as testing for microorganisms, preparation, sterilization, steps to inactivate or remove adventitious agents, preservation for storage, and removal from storage.

As used herein, a “culture,” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “mono-layer” by producing glycoprotein-like material that causes the cells to adhere to the plastic or glass of the Petri dish. A layer of culture medium, containing nutrients suitable for cell growth, is then placed on top of the mono-layer, and the culture is incubated to promote the growth of the cells.

As used herein, the term “subject” is used to mean any animal, preferably a mammal, including a human or non-human. The terms patient, subject, and individual are used interchangeably. None of the terms are to be interpreted as requiring the supervision of a medical professional (e.g., a doctor, nurse, physician's assistant, orderly, hospice worker).

“Substantially isolated” or “isolated” when used in the context of amniotic membranes means that the amniotic membrane is separated from most other non-amniotic membrane materials (e.g., red blood cells, blood vessels, and arteries) derived from the original source organism.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, the phrase “wherein the biological and structural integrity of the isolated amniotic membrane is substantially preserved” means that when compared to the biological activity and structural integrity of fresh amniotic membrane, the biological activity and structural integrity of the isolated amniotic membrane has only decreased by about 5%, about 10%, about 15%, about 20%, about 25%, about 30%, about 35%, about 40%, about 50%, about 60%.

The term “fresh amniotic membrane” refers to fresh amniotic membrane that is less than 10 days old following birth, and which is in substantially the same form as it was following birth.

As used herein, “biological activity” means the activity of polypeptides and polysaccharides. In some embodiments, the activity of polypeptides and polysaccharides found in amniotic membrane (and isolated amniotic membrane) is anti-inflammatory, anti-scarring, anti-angiogenic, or anti-adhesion. In some embodiments, the activity of polypeptides and polysaccharides found in amniotic membrane (and isolated amniotic membrane) promotion of wound healing.

Amniotic membranes are known to possess multiple layers—an epithelial layer, a basement membrane; a compact layer; a fibroblast layer; and a spongy layer. In general, it is known that amniotic membranes lack the immunogenic antigens HLA-A, B, or DR-reducing the risk of transplant rejection . Further, amniotic membranes are rich in cytokines such as IL-6, IL-8, EGF, TGF-.alpha., KGF, HGF, bFGF, TGF-.beta.1, TGF-.beta.2, PGE2, endothelin-1, leukotrienes, and lipoxin A [20]. These factors may be useful for the stimulation of regenerative activities. Furthermore, amniotic membranes have been shown to reduce inflammation, reduce angiogenesis, reduce scarring, and reduce adhesion [21-24]. Further, the basement membrane of the amniotic membranes serves as a natural niche for stem cells. Studies have shown that, wounds covered with amniotic membranes often display an increased rate of healing as compared to wounds covered with a tissue graft made of alternative materials [25-28]. In one embodiment, the invention uses amniotic membranes as starting material, which are isolated and cut into smaller pieces. As disclosed herein, in some embodiments, is a amniotic membrane particle, comprising: an isolated membrane that does not comprise a vein or an artery, does not possess infections such as active HIV-1, active HIV-2, active HTLV-1, active hepatitis B, active hepatitis C, active West Nile Virus, active cytomegalovirus, active human transmissible spongiform encephalopathy, or active treponema pallidum, wherein the natural structural integrity of the isolated amniotic membrane is preserved after initial procurement. In some embodiments said amniotic membrane is amniotic membrane derived from a placenta or from umbilical cord. Said placenta or cord blood is recovered from any suitable source (e.g., a hospital or tissue bank). Placenta or mbilical cord can be obtained from any mammal, such as a human, non-human primate, cow or pig. In some embodiments of the invention amniotic membrane is utilized as fresh tissues, in other embodiments, the amniotic membrane is utilized after freezing. In some embodiments it is kept at −80.degree. C. until donor and specimen eligibility has been determined. In other embodiments, the invention teaches storing the umbilical cord or placenta at −80.degree. All processing is done following Good Tissue Practices (GTP) to ensure that no contaminants are introduced into the umbilical cord or placental products. The tissue is tested for HIV-1, HIV-2, HTLV-1, hepatitis B and C, West Nile virus, cytomegalovirus, human transmissible spongiform encephalopathy (e.g., Creutzfeldt-Jakob disease) and treponema pallidum. Further, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and treponema pallidum results in the immediate quarantine and subsequent destruct of the tissue specimen.

In some embodiments, the umbilical cord or placenta is frozen. In some embodiments, the umbilical cord or placenta is not frozen. In some embodiments, substantially all of the blood is removed from the umbilical cord or placenta. In some embodiments, the umbilical cord tissue or placenta is washed with buffer with agitation to remove excess blood and tissue. In some embodiments, washing with agitation reduces the wash time. In some embodiments, the umbilical cord or placenta is washed with an isotonic buffer or tissue culture media. In some embodiments, the umbilical cord or placenta is washed with saline. In some embodiments, the umbilical cord or placenta is washed with PBS. In some embodiments, the umbilical cord or placenta is washed with PBS 1.times. In some embodiments, the umbilical cord or placenta is washed with a TRIS-buffered saline. In some embodiments, the umbilical cord or placenta is washed with a HEPES-buffered saline. In some embodiments, the umbilical cord is washed with Ringer's solution. In some embodiments, the umbilical cord or placenta is washed with Hartmann's solution. In some embodiments, the umbilical cord or placenta is washed with EBSS. In some embodiments, the umbilical cord or placenta is washed with HBSS. In some embodiments, the umbilical cord or placenta is washed with Tyrode's Salt Solution. In some embodiments, the umbilical cord is washed with Gey's Balanced Salt Solution. In some embodiments, the umbilical cord or placenta is washed with DMEM. In some embodiments, the umbilical cord or placenta is washed with EMEM. In some embodiments, the umbilical cord or placenta is washed with GMEM. In some embodiments, the umbilical cord or placenta is washed with RPMI.

In some embodiments, the umbilical cord or placenta is cut into multiple sections (e.g., using a scalpel). The size of the sections depends on the desired use of the product (e.g., tissue graft) derived from the umbilical cord.

In some embodiments, the umbilical cord is next contacted with a buffer to facilitate separation of the Wharton's Jelly and the amniotic membrane. In some embodiments, the umbilical cord is contacted with an isotonic buffer or tissue culture media. In some embodiments, the umbilical cord is contacted with saline. In some embodiments, the umbilical cord is contacted with PBS. In some embodiments, the umbilical cord is contacted with PBS 1.times.. In some embodiments, the umbilical cord is contacted with Ringer's solution. In some embodiments, the umbilical cord is contacted with Hartmann's solution. In some embodiments, the umbilical cord is contacted with a TRIS-buffered saline. In some embodiments, the umbilical cord is contacted with a HEPES-buffered saline. In some embodiments, the umbilical cord is contacted with EBSS. In some embodiments, the umbilical cord is contacted with HBSS. In some embodiments, the umbilical cord is contacted with Tyrode's Salt Solution. In some embodiments, the umbilical cord is contacted with Gey's Balanced Salt Solution. In some embodiments, the umbilical cord is contacted with EMEM. In some embodiments, the umbilical cord is contacted with DMEM. In some embodiments, the umbilical cord is contacted with GMEM. In some embodiments, the umbilical cord is contacted with RPMI.

In some embodiments, a section of the umbilical cord is then cut longitudinally (e.g., using a scalpel or scissors). In some embodiments, the section of the umbilical cord is not cut into halves. In some embodiments, the section of the umbilical cord is cut into two halves. Optionally, in some embodiments, additional cuts are made in the Wharton's Jelly to help flatten out the UC. In some embodiments, the cut umbilical cord tissue is optionally washed again with buffer to further remove excess blood and tissue. In some embodiments, the umbilical cord is fastened onto a substrate (e.g., a styrofoam board) using any suitable method (e.g., it is fastened with needles or pins (e.g., T pins)). In some embodiments, the umbilical cord is stabilized with a substrate (e.g., absorbent towel cloth, drape) In some embodiments, the umbilical cord is orientated such that the inside face of the umbilical cord (e.g., the face comprising the Wharton's Jelly) is facing up while the outside face is facing the substrate. Optionally, in some embodiments, one end of the umbilical cord is left free. Alternatively, in some embodiments, both ends of the umbilical cord are left free.

If the umbilical cord does not lay flat against the substrate, in some embodiments, additional cuts are made in the Wharton's Jelly.

In some embodiments, part or all of the Wharton's Jelly is removed from the amniotic membrane. The desired thickness of the tissue graft determines how much of the Wharton's Jelly is removed. In some embodiments, the Wharton's Jelly is peeled from the umbilical cord in layers (e.g., using a set of forceps, hemostats). In some embodiments, the Wharton's Jelly is cut away (e.g., shaved) from the umbilical cord in sections. In some embodiments, a rotoblator (i.e., a catheter attached to a drill with a diamond coated burr) is utilized to remove the Wharton's Jelly. In some embodiments, a liposuction machine is utilized to remove the Wharton's Jelly. In some embodiments, a liquid under high pressure is applied to remove the Wharton's Jelly. In some embodiments, a brush is utilized to remove the Wharton's Jelly (e.g., a mechanized brush rotating under high speed). In some embodiments, amniotic membrane is retrieved using a surgical dermatome.

In some embodiments, both ends of the umbilical cord are attached to the substrate. In some embodiments, only one end is attached to the substrate. See FIG. 3b. If one end of the umbilical cord is left free, in some embodiments, the free end of the umbilical cord is held (e.g., with a clamp, hemostats or a set of forceps (e.g., wide serrated tip forceps)) while part or all of the Wharton's Jelly is removed.

The umbilical cord comprises two arteries (the umbilical arteries) and one vein (the umbilical vein). In some embodiments, the vein and arteries are removed from the umbilical cord. In certain instances, the vein and arteries are surrounded (or suspended or buried) within the Wharton's Jelly. In some embodiments, the vein and arteries are removed concurrently with the removal of the Wharton's Jelly. In some embodiments, the vein and arteries are peeled (or pulled) from the umbilical cord (e.g., using a set of forceps). In some embodiments, the vein and arteries are cut away (e.g., shaved) from the umbilical cord in sections. In some embodiments, a rotoblator removes the vein and arteries concurrently with the Wharton's Jelly or from the placenta. In some embodiments, a liposuction machine is utilized to remove the vein and arteries concurrently with the Wharton's Jelly or placenta. In some embodiments, a vein stripper is utilized to remove the vein and arteries concurrently with the Wharton's Jelly. In some embodiments, a liquid under high pressure removes the vein and arteries concurrently with the Wharton's Jelly. In some embodiments, a brush removes the vein and arteries concurrently with the Wharton's Jelly. In some embodiments, a surgical dermatome removes the vein and arteries concurrently with the Wharton's Jelly. After substantially pure umbilical cord amniotic membrane has been obtained, the amniotic membrane is optionally washed with buffer to remove excess blood and tissue. In another embodiment, the umbilical cord is next contacted with a buffer to facilitate separation of the Wharton's Jelly and the amniotic membrane. In some embodiments, the umbilical cord is contacted with an isotonic buffer or tissue culture media. In some embodiments, the umbilical cord is contacted with saline. In some embodiments, the umbilical cord is contacted with PBS. In some embodiments, the umbilical cord is contacted with PBS 1.times.. In some embodiments, the umbilical cord is contacted with Ringer's solution. In some embodiments, the umbilical cord is contacted with Hartmann's solution. In some embodiments, the umbilical cord is contacted with a TRIS-buffered saline. In some embodiments, the umbilical cord is contacted with a HEPES-buffered saline. In some embodiments, the umbilical cord is contacted with EBSS. In some embodiments, the umbilical cord is contacted with HBSS. In some embodiments, the umbilical cord is contacted with Tyrode's Salt Solution. In some embodiments, the umbilical cord is contacted with Gey's Balanced Salt Solution. In some embodiments, the umbilical cord is contacted with EMEM. In some embodiments, the umbilical cord is contacted with DMEM. In some embodiments, the umbilical cord is contacted with GMEM. In some embodiments, the umbilical cord is contacted with RPMI.

In some embodiments, the amniotic membrane is cut into sheets are in any suitable shape (e.g., a square, a circle, a triangle, a rectangle). The size of the amniotic membrane depends on the desired use. In some embodiments, the amniotic membrane is contacted with a buffer to remove substantially all remaining red blood cells. In some embodiments, the amniotic membrane is contacted with an isotonic buffer. In some embodiments, the amniotic membrane is contacted with saline. In some embodiments, the amniotic membrane is contacted with PBS or other solutions such as Ringer's solution, Hartmann's solution, TRIS-buffered saline, HEPES-buffered saline, HBSS, Tyrode's salt Solution, Gey's Balanced Salt Solution, DMEM, EMEM, and RPMI.

The amniotic membrane can be used to prepare the composition. Amniotic membrane preparations can include components or portions purified from or extracted from intact amniotic membrane, amniotic membrane stromal matrix, HA, amniotic membrane jelly, and inter-alpha trypsin inhibitor (HA-ITI)). If desired, certain components of the amniotic membrane preparation can be isolated from the preparation at any time during the process. For example, an extract enriched for a specific protein or set of amniotic membrane proteins can be isolated from the preparation. After homogenization of the tissue, the larger particles can be separated out, or they can be left in the preparation. The preparation can be dried, if desired. The compositions can also be obtained from amniotic membrane jelly. Amniotic membrane jelly can be obtained from the fresh amniotic membrane tissue, or can be obtained before or after the freezing process. The amniotic membrane jelly can be frozen, and can also be freeze-ground following the procedure for amniotic membrane preparations as described herein. The jelly can be centrifuged, and can also be lyophilized.

In additional embodiments, a composition made substantially from the stromal layer is prepared. To prepare this composition, the stromal layer is separated from the layer of fresh, frozen, thawed, or otherwise treated amniotic membrane membrane. The stromal removal can occur, for example, by enzymatic methods, mechanical methods, or by other means. The stromal layer material can be fresh or frozen. The stromal material can be ground or freeze-ground following the procedure for amniotic membrane preparations as described herein. If desired, the stromal matrix material can be centrifuged, and can also be lyophilized.

The tissue can be frozen prior to the grinding process. The freezing step can occur by any suitable cooling process. For example, the tissue can be flash-frozen using liquid nitrogen. Alternatively, the material can be placed in an isopropanol/dry ice bath or can be flash-frozen in other coolants. Commercially available quick freezing processes can be used. Additionally, the material can be placed in a freezer and allowed to equilibrate to the storage temperature more slowly, rather than being flash-frozen. The tissue can be stored at any desired temperature. For example, −20.degree. C. or −80.degree. C. or other temperatures can be used for storage. Pulverizing the tissue while frozen, rather than grinding the tissue prior to freezing, is one optional method for preparing the tissue. Alternatively, fresh, partially thawed, or thawed tissue can be used in the grinding step. The tissue (fresh, frozen, or thawed) can then be sliced into pieces of a desired size with a suitable device, such as a scalpel, then ground to fine particles using a BioPulverizer (Biospec Products, Inc., Bartlesville, Okla.) or other suitable devices, and homogenized with a homogenization device such as a Tissue Tearor (Biospec Products, Inc., Dremel, Wis., in a suitable solution. Exemplary solutions include but are not limited to phosphate buffered saline (PBS), DMEM, NaCl solution, and water. The pH of the solution can be adjusted as needed. In some embodiments, the pH range is from about 5.5 or 6.0 to about 8.5.In some embodiments, the frozen tissue is ground in a solution having a pH of between about 6.3, about 6.6, or about 7.0 to about 7.4, about 7.6, or about 7.8.

Once amniotic membranes are isolated, generation of particulates or smaller pieces of amniotic membrane may be performed by various means. In one particular embodiment amniotic membranes may be morselized by various means known in the art. Other means of generation of amniotic membrane particles may be by cutting, shearing, forcing through a mesh, or by other mechanical or physical means. Polymers may be added to the cells or morselized tissue containing cells, said polymers may be added to provide a scaffold to enhance retention in a particular site, or to augment viability of cells. Specific polymers for use in forming polymeric components in accordance with the invention may be selected, for example, from one or more suitable members of the following, among others: polycarboxylic acid homopolymers and copolymers including polyacrylic acid, polymethacrylic acid, ethylene-methacrylic acid copolymers and ethylene-acrylic acid copolymers, where some of the acid groups can be neutralized with either zinc or sodium ions (commonly known as ionomers); acetal homopolymers and copolymers; acrylate and methacrylate homopolymers and copolymers (e.g., n-butyl methacrylate); cellulosic homopolymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene homopolymers and copolymers; polyimide homopolymers and copolymers such as polyether block imides, polyamidimides, polyesterimides, and polyetherimides; polysulfone homopolymers and copolymers including polyarylsulfones and polyethersulfones; polyamide homopolymers and copolymers including nylon 6,6, nylon 12, polycaprolactams, polyacrylamides and polyether block amides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); homopolymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene (SIBS) block copolymers such as those disclosed in U.S. Pat. No. 6,545,097 to Pinchuk), poly[(styrene-co-p-methylstyrene)-b-isobutylene-b-(styrene-co-p-methylstyrene)] (SMIMS) triblock copolymers described in S. J. Taylor et al., Polymer 45 (2004) 4719-4730; polyphosphonate homopolymers and copolymers; polysulfonate homopolymers and copolymers, for example, sulfonated vinyl aromatic polymers and copolymers, including block copolymers having one or more sulfonated poly(vinyl aromatic) blocks and one or more polyalkene blocks, for example, sulfonated polystyrene-polyolefin-polystyrene triblock copolymers such as the sulfonated SEBS copolymers described in U.S. Pat. No. 5,840,387, and sulfonated versions of SIBS and SMIMS, which polymers may be sulfonated, for example, using the processes described in U.S. Pat. No. 5,840,387 and U.S. Pat. No. 5,468,574, among other sulfonated block copolymers; polyvinyl ketones, polyvinylcarbazoles, and polyvinyl esters such as polyvinyl acetates; polybenzimidazoles; polyalkyl oxide homopolymers and copolymers including polyethylene oxides (PEO); polyesters including polyethylene terephthalates and aliphatic polyesters such as homopolymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether homopolymers and copolymers including polyarylethers such as polyphenylene ethers, polyether ketones, polyether ether ketones; polyphenylene sulfides; polyisocyanates; polyolefin homopolymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated homopolymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone homopolymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; polyamine and polyimine homopolymers and copolymers; biopolymers, for example, polypeptides including anionic polypeptides such as polyglutamate and cationic polypeptides such as polylysine, proteins, polysaccharides, and fatty acids (and esters thereof), including fibrin, fibrinogen, collagen, elastin, chitosan, gelatin, starch, glycosaminoglycans such as hyaluronic acid; as well as further copolymers of the above.

Specific polymers that are of use in the practice of the invention are biodegradable polymers, not necessarily exclusive of those set forth above, may be selected from suitable members of the following, among many others: (a) polyester homopolymers and copolymers such as polyglycolide, poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide) (PLGA), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], and poly(sebacic acid-co-fumaric acid), among others, (b) poly(ortho esters) such as those synthesized by copolymerization of various diketene acetals and diols, among others, (c) polyanhydrides such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others; and (d) amino-acid-based polymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine), and tyrosine-, leucine- and lysine-based polyester-amides; specific examples of tyrosine-based polymers include includes polymers that are comprised of a combination of desaminotyrosyl tyrosine hexyl ester, desaminotyrosyl tyrosine, and various di-acids, for example, succinic acid and adipic acid, more specifically tyrosine-derived ester-amides such as the TyRx 2,2 family of polymers, available from TyRx Pharma, Inc., Monmouth Junction, N.J., USA, among others.

The antibiotic and/or antimycotic pretreatment of cellular aggregates alone, or cellular aggregates associated with polymers may be performed using various antibiotics known in the art. Examples of antibiotics useful for the practice of the invention include: antibiotic may be any of the following, alone or in combination: an aminoglycoside, for example amikacin, gentamicin, kanamycin, neomycin, netilmicin, streptomycin or tobramycin; a carbacephem, for example loracarbef; a carbapenem, for example ertapenem, imipenem/cilastatin or meropenem; a cephalosporin (first generation), for example cefadroxil, cefazohn or cephalexin; a cephalosporin (second generation), for example cefaclor, cefamandole, cefoxitin, cefprozil or cefuroxime; a cephalosporin (third generation), for example cefixime, cefdinir, cefditoren, cefoperazone, cefotaxime, cefpodoxime, ceftazidime, ceftibuten, ceftizoxime or ceftriaxone; a cephalosporin (fourth generation), for example cefepime; a glycopeptide, for example teicoplanin or vancomycin; a penicillin, for example amoxicillin, ampicillin, azlocillin, carbenicillin, cloxacillin, dicloxacillin, flucloxacillin, mezlocillin, nafcillin, penicillin, piperacillin or ticarcillin; a (poly)peptide, for example bacitracin, colistin or polymyxin B; a quinolone, for example ciprofloxacin, enoxacin, gatifloxacin, levofloxacin, lomefloxacin, moxifloxacin, norfloxacin, ofloxacin or trovafloxacin; a sulfonamide, for example mafenide, prontosil, sulfacetamide, sulfamethizole, sulfanamide, sulfasalazine, sulfisoxazole, trimethoprim or trimethoprim-sulfamethoxazole; a tetracycline, for example demeclocycline, doxycycline, minocycline, oxytetracycline or tetracycline;—any nother antibiotic, for example chloramphenicol, clindamycin, ethambutol, fosfomycin, furazolidone, isoniazid, linezohd, metronidazole, nitrofurantoin, pyrazinamide, quinupristin/dalfopristin, rifampin or spectinomycin. Additionally, antimycotics useful for the practice of the invention include nystatin, clotrimazole, miconazole, econazole, ketoconazole, bifonazole, and combinations of imidazole and triazole derivatives, ciclopirox, terbinafine, fluconazole, and amorolfine (and pharmaceutically acceptable salts and derivatives thereof);

Other tissues besides amniotic membrane may be treated in order to modulate buoyancy, for example, morselized bone tissue, pancreatic islet tissues, hepatic tissues, or other tissues in which injectable forms are desired.

### EXAMPLE

Antibiotic-antimycotic cocktails were prepared at 100%, 75%, 50%, and 25% strengths. Three pieces of amnion were non-aseptically processed in the general lab benchtop area. The antibiotic stock solution (100%) was comprised of Gentamicin Sulfate at 50 ug/ml, Penicillin at 100 U/ml, Streptomycin at 100 ug/ml, and Amphotericin B at 250 ng/ml. Amniotic membrane tissue was cut in four pieces and placed in the cocktail. Tissue was incubated in the cocktail on an orbital shaker for one hour. After one hour, the tissue was rinsed three times in PBS. After the final rinse, the tissue was centrifuged for 5 minutes at 800 g. Tissue was milled to observe buoyancy in PBS. Neutral buoyancy was observed using the 50% stock of Antibiotic-antimycotic-gentamicin concentration. The 100% and 75% stock produced tissue that floated on the surface of the PBS, while the 25% stock produced tissue that settled rapidly to the bottom of the tube.

### REFERENCES

1. Stambler, I., The unexpected outcomes of anti-aging, rejuvenation, and life extension studies: an origin of modern therapies. Rejuvenation Res, 2014. 17(3): p. 297-305.
2. Letteh, H., [On the problem of Niehans' cellular therapy]. Arzneimittelforschung, 1954. 4(8): p. 484-5.
3. de Ridder, M., et al., [2 cases of death following cell therapy]. Dtsch Med Wochenschr, 1987. 112(25): p. 1006-9.
4. Thomas, E. D., Bone marrow transplantation: a review. Semin Hematol, 1999. 36(4 Suppl 7): p. 95-103.
5. Caplan, A. I., Why are MSCs therapeutic? New data: new insight. J Pathol, 2009. 217(2): p. 318-24.
6. Ricordi, C., et al., NIH-sponsored Clinical Islet Transplantation Consortium Phase 3 Trial: Manufacture of a Complex Cellular Product at Eight Processing Facilities. Diabetes, 2016.
7. Jahnisch, H., et al., Dendritic cell-based immunotherapy for prostate cancer. Clin Dev Immunol, 2010. 2010: p. 517493.

8. Harzstark, A. L. and E. J. Small, Sipuleucel-T for the treatment of prostate cancer. Drugs Today (Barc), 2008. 44(4): p. 271-8.
9. Ten Brinke, A., et al., Clinical Use of Tolerogenic Dendritic Cells-Harmonization Approach in European Collaborative Effort. Mediators Inflamm, 2015. 2015: p. 471719.
10. Mok, M. Y., Tolerogenic dendritic cells: role and therapeutic implications in systemic lupus erythematosus. Int J Rheum Dis, 2015. 18(2): p. 250-9.
11. Daniyan, A. F. and R. J. Brentjens, At the bench: chimeric antigen receptor (CAR) T cell therapy for the treatment of B cell malignancies. J Leukoc Biol, 2016.
12. Holzinger, A., M. Barden, and H. Abken, The growing world of CAR T cell trials: a systematic review. Cancer Immunol Immunother, 2016. 65(12): p. 1433-1450.
13. Gad, A. Z., S. El-Naggar, and N. Ahmed, Realism and pragmatism in developing an effective chimeric antigen receptor T-cell product for solid cancers. Cytotherapy, 2016. 18(11): p. 1382-1392.
14. Raines, A., et al., Efficacy of Particulate Amniotic Membrane and Umbilical Cord Tissues in Attenuating Cartilage Destruction in an Osteoarthritis Model. Tissue Eng Part A, 2016.
15. Hanselman, A. E., J. E. Tidwell, and R. D. Santrock, Cryopreserved human amniotic membrane injection for plantar fasciitis: a randomized, controlled, double-blind pilot study. Foot Ankle Int, 2015. 36(2): p. 151-8.
16. Zelen, C. M., A. Poka, and J. Andrews, Prospective, randomized, blinded, comparative study of injectable micronized dehydrated amniotic/chorionic membrane allograft for plantar fasciitis—a feasibility study. Foot Ankle Int, 2013. 34(10): p. 1332-9.
17. Cheng, A. M., et al., Morselized Amniotic Membrane Tissue for Refractory Corneal Epithelial Defects in Cicatricial Ocular Surface Diseases. Transl Vis Sci Technol, 2016. 5(3): p. 9.
18. Swan, J., Use of Cryopreserved, Particulate Human Amniotic Membrane and Umbilical Cord (AM/UC) Tissue: A Case Series Study for Application in the Healing of Chronic Wounds. Surg Technol Int, 2014. 25: p. 73-8.
19. Bonci, P., P. Bonci, and A. Lia, Suspension made with amniotic membrane: clinical trial. Eur J Ophthalmol, 2005. 15(4): p. 441-5.
20. Tan, J. L., et al., Amnion Epithelial Cells Promote Lung Repair via Lipoxin A4. Stem Cells Transl Med, 2016.
21. Sant'Anna, L. B., et al., Anti-fibrotic effects of human amniotic membrane transplantation in established biliary fibrosis induced in rats. Cell Transplant, 2016.
22. Nassif, J., et al., Effect of the Mode of Application of Cryopreserved Human Amniotic Membrane on Adhesion Formation after Abdomino-Pelvic Surgery in a Mouse Model. Front Med (Lausanne), 2016. 3: p. 10.
23. Barbuto, R. C., et al., Use of the amniotic membrane to cover the peritoneal cavity in the reconstruction of the abdominal wall with polypropylene mesh in rats. Rev Col Bras Cir, 2015. 42(1): p. 49-55.
24. Kirsch, D., et al., Amniotic membrane for reducing the formation of adhesions in strabismus surgery: experimental study in rabbits. J Pediatr Ophthalmol Strabismus, 2014. 51(6): p. 341-7.
25. DiDomenico, L. A., et al., Aseptically Processed Placental Membrane Improves Healing of Diabetic Foot Ulcerations: Prospective, Randomized Clinical Trial. Plast Reconstr Surg Glob Open, 2016. 4(10): p. e1095.
26. Miranda, E. P. and A. Friedman, Dehydrated Human Amnion/Chorion Grafts May Accelerate the Healing of Ulcers on Free Flaps in Patients With Venous Insufficiency and/or Lymphedema. Eplasty, 2016. 16: p. e26.
27. Abdo, R. J., Treatment of diabetic foot ulcers with dehydrated amniotic membrane allograft: a prospective case series. J Wound Care, 2016. 25 Suppl 7: p. S4-9.
28. Raphael, A., A single-centre, retrospective study of cryopreserved umbilical cord/amniotic membrane tissue for the treatment of diabetic foot ulcers. J Wound Care, 2016. 25 Suppl 7: p. S10-7.

1. A method for modulating buoyancy of a dissected tissue in which said dissected tissue is contacted with an agent selected from the group consisting of: an antibiotic, a plurality of antibiotics, and a derivative of an antibiotic, at a sufficient concentration and timepoint to achieve a desired buoyancy.

2. The method of claim 1, wherein said dissection comprises morselization of said tissue.

3. The method of claim 1, wherein said dissection comprises cutting of said tissue.

4. The method of claim 1, wherein said dissection comprises grinding of said tissue.

5. The method of claim 1, wherein said dissection comprises shearing of said tissue.

6. The method of claim 1, wherein said dissection comprises sonication of said tissue.

7. The method of claim 1, wherein said dissection comprises application of mechanical pressure of sufficient strength in order to cause dissociation of said tissue into parts smaller than the original tissue.

8. The method of claim 1, wherein said buoyancy is modulated in a manner to prevent said dissected tissue from gravity associated settling.

9. The method of claim 1, wherein said buoyancy is modulated in a manner to allow said dissected tissue to float.

10. The method of claim 1, wherein said dissected tissue is a tissue in which an injectable form is desired.

11. The method of claim 1, wherein said dissected tissue is pancreatic tissue.

12. The method of claim 1, wherein said dissected tissue is hepatic tissue.

13. The method of claim 1, wherein said dissected tissue is adipose tissue.

14. The method of claim 1, wherein said dissected tissue is placental tissue.

15. The method of claim 1, wherein said dissected tissue is amniotic tissue.

16. The method of claim 1, wherein said dissected tissue is amniotic membrane.

17. The method of claim 1, wherein said dissected tissue is Wharton's Jelly.

18. The method of claim 16, wherein said amniotic membrane is dehydrated.

19. The method of claim 16, wherein said amniotic membrane has previously been cryopreserved.

20. The method of claim 16, wherein said amniotic membrane has previously been terminally sterilized.

21. The method of claim 16, wherein said amniotic membrane has previously been terminally sterilized and cryopreserved.

22. The method of claim 1, wherein said antibiotic is an antibiotic-antimycotic cocktail.

23. The method of claim 22, wherein said antibiotic-antimycotic cocktail is comprised of gentamicin sulfate, penicillin, streptomycin, and amphotericin B.

24. The method of claim 23, wherein said concentration of gentamicin sulfate is between 5 ug/ml and 500 ug/ml, wherein said concentration of penicillin is 10-1000 U/ml, wherein said concentration of streptomycin is 10-1000 ug/ml, wherein said concentration of amphotericin B is 25-2500 ng/ml.

25. The method of claim 24, wherein said concentration of gentamicin sulfate is between 25 ug/ml and 250 ug/ml, wherein said concentration of penicillin is 50-500 U/ml, wherein said concentration of streptomycin is 10-1000 ug/ml, wherein said concentration of amphotericin B is 25-2500 ng/ml.
